(19) United States
(12) Patent Application Publication
(10) Pub. No.: US 2026/0061031 A1
(43) Pub. Date: Mar. 5, 2026

(54) METHODS AND PHARMACEUTICAL COMPOSITIONS FOR THE TREATMENT OF DILATED CARDIOMYOPATHY

(71) Applicants: INSTITUT NATIONAL DE LA SANTÉ ET DE LA RECHERCHE MÉDICALE, PARIS (FR); UNIVERSITÉ DE ROUEN NORMANDIE, Mont-Saint Aignan (FR)

(72) Inventors: Sahil ADRIOUCHI, ROUEN CEDEX 1 (FR); Theo LEMARCIS, ROUEN CEDEX (FR); Virginie TARDIF SAN MARTIN, ROUEN CEDEX 1 (FR)

(21) Appl. No.: 19/105,235

(22) PCT Filed: Sep. 13, 2023
(86) PCT No.: PCT/EP2023/075116

§ 371 Date: Feb. 20, 2025

(30) Foreign Application Priority Data
Sep. 14, 2022 (EP) 22306349.6

(51) Int. Cl. A61K 38/17; A61K 39/395; A61K 45/06; A61P 9/04; G01N 33/50

(52) U.S. Cl. CPC A61K 38/1774; A61K 39/3955; A61K 45/06; A61P 9/04; G01N 33/5023; G01N 2333/70521

(57) ABSTRACT

Co-signaling immunotherapy, via immune checkpoint inhibitors (ICIs), such as ipilimumab (anti-CTLA-4) or nivolumab (anti-PD-1), has revolutionized cancer treatment. However, reinvigorating tumor-infiltrating T cell cytotoxicity has revealed cardiac toxicity in a subset of patients. The inventors found lower transcriptomic expression of CTLA-4 in eccentric hypertrophic cardiomyopathy model, associated with higher cardiac total IgG amount and less B cell infiltration, compared to concentric hypertrophic cardiomyopathy. The present invention relates to a method of treating dilated cardiomyopathy (DCM) in a subject in need thereof comprising a step of administering to said subject a therapeutically effective amount of a cytotoxic T-lymphocyte-associated protein (CTLA-4) molecule.

### FIELD OF THE INVENTION

The present invention is in the field of medicine, in particular cardiology.

### BACKGROUND OF THE INVENTION

Worldwide estimates of people suffering from HF are about 26 million and their significant health, economic and healthcare burdens are expected to increase over the next decade as HF patient numbers rise owing to ageing of the population41. Current treatment strategies are insufficient, and across the globe 17-45% of hospitalized HF patients die within 1 year, and the majority within 5 years.

Immune checkpoint inhibitors (ICIs), such as ipilimumab (anti-CTLA-4) or nivolumab (anti-PD-1), targeting immune cell co-signaling molecules, have emerged in oncology to boost anti-tumoral immune responses. However, ICIs can cause serious and even fatal immune related-adverse events (irAEs) due to deregulated immunity, including significant cardiovascular adverse events (CV-irAEs), mainly myocarditis, but also pericardial diseases and vasculitis, highlighting the need for reappraising the role of immune responses/inflammation in heart failure (HF). Indeed, despite remarkable progress in terms of early diagnosis and prevention of cardiovascular diseases (CVD), chronic HF remains a severe and increasingly common sequela to myocardial infarction (MI), myocarditis and other cardiomyopathies including non-ischemic hypertrophic (HCM) and dilated (DCM) cardiomyopathies. Cardiac inflammation, together with edema and fibrosis, are common traits in many CVDs.

This highlights the need for innovative targeted therapy to improve HF outcomes. In parallel, breakthrough innovation in oncology with immunotherapy using ICI has unveiled cardiac toxicity, which while cases currently are rare, may increase in next decades due to the expected common use of such immunotherapy, highlighting the need for better understanding of cardiac co-signaling pathways. Here, based on in vivo and ex vivo molecular characterization of cardiac co-signaling, it aims to unravel the interconnection between cardiac inflammation, lymphatic dysfunction, and fibrosis during HF development.

The inventors identify and validate innovative therapeutic strategies to restore cardiac lymphatic function, re-equilibrate immune responses, reduce cardiac fibrosis and treat dilated cardiomyopathy (DCM).

### SUMMARY OF THE INVENTION

The present invention is defined by the claims. In particular, the present invention relates to methods of treating dilated cardiomyopathy (DCM).

### DETAILED DESCRIPTION OF THE INVENTION

Co-signaling immunotherapy, via immune checkpoint inhibitors (ICIs), such as ipilimumab (anti-CTLA-4) or nivolumab (anti-PD-1), has revolutionized cancer treatment. However, reinvigorating tumor-infiltrating T cell cytotoxicity has revealed cardiac toxicity in a subset of patients, uncovering still not fully deciphered cardiac immune tolerance mechanisms. In this context, the inventors aim to study the pathogenic deregulation of co-signalling pathways and its impact on the cardiac immune response in cardiovascular diseases, which may affect lymphatic and vascular remodeling in the heart, leading to poor resolution of cardiac inflammation and edema, and subsequently HF progression.

Left ventricular pressure-overload model in mice is induced by surgical transversal aortic constriction, leading to the development of either concentric hypertrophic cardiomyopathy or eccentric hypertrophic cardiomyopathy, depending on murine strain. Tissular and cellular snapshot of co-signaling pathways during cardiac remodeling has been evaluated by transcriptomic tools (qPCR, RNAseq).

The inventors found lower transcriptomic expression of CTLA-4 in eccentric hypertrophic cardiomyopathy model, associated with higher cardiac total IgG amount and less B cell infiltration, compared to concentric hypertrophic cardiomyopathy.

### METHOD OF TREATING DILATED CARDIOMYOPATHY

Accordingly, in a first aspect, the present invention relates to a method of treating dilated cardiomyopathy (DCM) in a subject in need thereof comprising a step of administering to said subject a therapeutically effective amount of a cytotoxic T-lymphocyte-associated protein 4 (CTLA-4) molecule.

As used herein, the terms “subject” or “patient” denote a mammal, such as a rodent, a feline, a canine, and a primate. Particularly, the subject according to the invention is a human. Particularly, the subject according to the invention is a child, a teenager, an adult or an elderly person. In some embodiments, the subject is more than 15 years old. In some embodiments, the subject is more than 20 years old. In some embodiments, the subject is more than 25 years old. In some embodiments, the subject is more than 30 years old. In some embodiments, the subject is more than 35 years old.

As used herein, the term “heart failure” (HF) has its general meaning in the art and embraces congestive heart failure and/or chronic heart failure. Functional classification of heart failure is generally done by the New York Heart Association Functional Classification (Criteria Committee, New York Heart Association. Diseases of the heart and blood vessels. Nomenclature and criteria for diagnosis, 6^{th} ed. Boston: Little, Brown and co, 1964; 114). This classification stages the severity of heart failure into 4 classes (I-IV). The classes (I-IV) are: Class I: no limitation is experienced in any activities; there are no symptoms from ordinary activities; Class II: slight, mild limitation of activity; the patient is comfortable at rest or with mild exertion; Class III: marked limitation of any activity; the patient is comfortable only at rest; Class IV: any physical activity brings on discomfort and symptoms occur at rest.

As used herein, the term “cardiac toxicity” refers to a side effect of cancer treatment that leads to damage to the heart muscle or valves. Chemotherapy, radiation, immunotherapy (i.g. Immune checkpoint inhibitor) can contribute to cardiac toxicity, depending on the type of medication(s) used and where radiation treatment was given. Cardiac toxicity can happen as a late effect of treatment, occurring months to years after treatment has ended. Cancer treatments not only kill cancer cells but also damage or kill healthy cells. When these cells are in or around the heart, cardiac toxicity occurs. There are many types of cardiac injury that can happen, for example: cardiomyopathy, myocarditis, pericarditis, acute coronary syndromes, congestive heart failure (CHF).

In some embodiment, the present invention aims to reduce cardiac fibrosis.

As used herein, the term “cardiac fibrosis” has its general meaning in the art and refers to a condition characterized by the excessive production and deposition of ECM proteins into the myocardium which leads to normal tissue architecture disruption, reduced tissue compliance, mechanical and electrical dysfunction. Cardiac fibrosis arises from aging, exposure to certain drugs, or in response to various heart diseases, such as myocardial infarction and hypertension. Following acute myocardial infarction, sudden loss of a large number of cardiomyocytes triggers an inflammatory reaction, ultimately leading to replacement of dead myocardium with a collagen-based scar. Several other pathophysiologic conditions induce more insidious interstitial and perivascular deposition of collagen, in the absence of completed infarction. Aging is associated with progressive fibrosis that may contribute to the development of diastolic HF in elderly patients. Pressure overload, induced by hypertension or aortic stenosis, results in extensive cardiac fibrosis that is initially associated with increased stiffness and diastolic dysfunction. Volume overload due to valvular regurgitant lesions may also result in cardiac fibrosis. Hypertrophic cardiomyopathy and post-viral dilated cardiomyopathy are also often associated with the development of significant cardiac fibrosis. Moreover, a variety of toxic insults (such as alcohol or anthracyclines) and metabolic disturbances (such as diabetes and obesity) induce cardiac fibrosis.

In some embodiment, the present invention aims to restore cardiac lymphatic function.

As used herein, the term “cardiac lymphatic” refers to a dynamic range of fluid uptake and transport that is linked to cardiac contractility and heart rate. Cardiac lymphatics undergo substantial remodelling in several cardiovascular diseases, which can alter the lymphatic drainage capacity in the heart.

In some embodiment, the method of the invention will improve lymphatic, vascular and cardiac remodeling in the heart and thus cardiomyopathy.

The present invention also relates to a method for modulating cardiac remodeling in a subject in need thereof comprising a step of administering to said subject a therapeutically effective amount of a cytotoxic T-lymphocyte-associated protein 4 (CTLA-4) molecule.

As used herein, the “cardiac remodeling” refers to a group of molecular, cellular and interstitial changes that manifest clinically as changes in size, mass, geometry and function of the heart after injury. In some models, alterations in geometry, including changes in the wall thickness, cavity diameter, and normal configuration of the left ventricle (from elliptical to spherical), may lead to functional consequences. The process results in poor prognosis because of its association with ventricular dysfunction and malignant arrhythmias. Cardiac dysfunction is the main consequence of cardiac remodeling, which consists of a pathophysiological substrate for the onset and progression of ventricular dysfunction.

As used herein, the term “cardiomyopathy” refers to a group of diseases that affect the heart muscle. Early on there may be few or no symptoms. As the disease worsens, shortness of breath, feeling tired, and swelling of the legs may occur, due to the onset of heart failure. An irregular heart beat and fainting may occur. Those affected are at an increased risk of sudden cardiac death. Types of cardiomyopathy include hypertrophic cardiomyopathy, dilated cardiomyopathy, restrictive cardiomyopathy, arrhythmogenic right ventricular dysplasia, and Takotsubo cardiomyopathy (broken heart syndrome).

As used herein, the term “hypertrophic cardiomyopathy” (HCM) has its general meaning in the art and is defined clinically as unexplained left ventricular (LV) hypertrophy in the absence of known causes such as pressure overload, systemic diseases, or infiltrative processes. The phenotypic hallmark of HCM is myocardial hypercontractility accompanied by reduced LV compliance, reflected clinically as reduced ventricular chamber size, often supranormal ejection fraction, increased wall thickness, and diastolic dysfunction. Some of the symptoms and signs that HCM patients have include, but are not limited to, shortness of breath (especially during exercise), chest pain (especially during exercise), fainting (especially during or just after exercise), sensation of rapid, fluttering or pounding heartbeats, and heart murmur.

In some embodiments, the cardiomyopathy is dilated cardiomyopathy.

As used herein, the term “dilated cardiomyopathy” (DCM) has its general meaning in the art and refers to a condition in which the heart becomes enlarged and cannot pump blood effectively. Symptoms vary from none to feeling tired, leg swelling, and shortness of breath. It may also result in chest pain or fainting. Complications can include heart failure, heart valve disease, or an irregular heartbeat. The progression of heart failure is associated with left ventricular remodeling, which manifests as gradual increases in left ventricular end-diastolic and end-systolic volumes, wall thinning, and a change in chamber geometry to a more spherical, less elongated shape. This process is usually associated with a continuous decline in ejection fraction.

In some embodiment, the present invention relates to a CTLA-4 molecule for use in the treatment of dilated cardiomyopathy.

As used herein, the term “treatment” or “treat” refer to both prophylactic or preventive treatment as well as curative, improving the patient's condition or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., daily, weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the terms “CTLA-4″ or ”CTLA4″ (cytotoxic T-lymphocyte-associated protein 4), also known as CD152 (cluster of differentiation 152), is a protein receptor that functions as an immune checkpoint and downregulates immune responses. CTLA-4 is constitutively expressed in regulatory T cells. CTLA-4 is a member of the immunoglobulin superfamily that is expressed by activated T cells and transmits an inhibitory signal to T cells. CTLA-4 is homologous to the T-cell co-activatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. CTLA-4 transmits an inhibitory signal to T cells, whereas CD28 transmits an activatory signal. CTLA-4 is also found in regulatory T cells (Tregs) and contributes to their inhibitory function. T cell activation through the T cell receptor and CD28 leads to increased expression of CTLA-4. Nucleic acid sequence (Gene ID: 1493) and amino acid sequence of (UniProt: P16410) of human CTLA-4 in particular are described in the art.

In some embodiment, CLTA-4 molecule promotes the CLTA-4 actions that restore cardiac lymphatic function, re-equilibrate immune responses and reduce cardiac fibrosis.

The CLTA-4 molecule is a small organic molecule, an aptamer or a polypeptide.

As used herein the term “aptamers” refers to a class of molecule that represents an alternative to antibodies in term of molecular recognition. Aptamers are oligonucleotide or oligopeptide sequences with the capacity to recognize virtually any class of target molecules with high affinity and specificity.

As used herein the term “small organic molecule” refers to a molecule of a size comparable to those organic molecules generally used in pharmaceuticals. The term excludes biological macro molecules (e.g. proteins, nucleic acids, etc.). Typically, small organic molecules range in size up to about 5000 Da, more preferably up to 2000 Da, and most preferably up to about 1000 Da.

In some embodiment, CLTA-4 molecule is Abatacept.

As used herein the term “Abatacept” also known as Orencia is a medication which interfere with the immune activity of T cells. It is a modified antibody. Abatacept is a fusion protein composed of the Fc region of the immunoglobulin IgGI fused to the extracellular domain of CTLA-4. In order for a T cell to be activated and produce an immune response, an antigen-presenting cell must present two signals to the T cell. One of those signals is the major histocompatibility complex (MHC), combined with the antigen, and the other signal is the CD80 or CD86 molecule (also known as B7-1 and B7-2). Abatacept binds to the CD80 and CD86 molecule, and prevents the second signal. Without the second signal, the T cell can't be activated. Abatacept is having the following CAS number: 332348-12-6.

In some embodiment, CLTA-4 molecule is Belatacept.

As used herein the term “Belatacept” also known as Nulojix, is a fusion protein composed of the Fc fragment of a human IgG1 immunoglobulin linked to the extracellular domain of CTLA-4, which is a molecule crucial in the regulation of T cell co-stimulation, selectively blocking the process of T-cell activation. It is intended to provide extended graft and transplant survival while limiting the toxicity generated by standard immune suppressing regimens, such as calcineurin inhibitors. It differs from abatacept (Orencia) by only two amino acids. Belatacept is having the following CAS number: 706808-37-9.

As used herein, the term “preventing” intends characterizing a prophylactic method or process that is aimed at delaying or preventing the onset of a disorder or condition to which such term applies.

As used herein the terms “administering” or “administration” refer to the act of injecting or otherwise physically delivering a substance as it exists outside the body (e.g. CTLA-4 molecule) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Accordingly, the subject is administered with a pharmaceutical composition comprising the CLTA-4 molecule as active principle and at least one pharmaceutically acceptable excipient. As used herein the term “active principle” or “active ingredient” are used interchangeably. The active principle is used to alleviate, treat or prevent a medical condition or disease. By the term “pharmaceutically acceptable excipient” herein, it is understood a carrier medium which does not interfere with the effectiveness of the biological activity of the active ingredient(s) and which is not excessively toxic to the host at the concentration at which it is administered. Said excipients are selected, depending on the pharmaceutical form and the desired method of administration, from the usual excipients known by a person skilled in the art. In some embodiments, the pharmaceutical composition of the present invention does not comprise a second active principle.

### Combined Preparation

The molecule of CTLA-4 as described above is combined with classical treatments.

Accordingly, the invention relates to i) molecule of CTLA-4 and ii) a classical treatment used as a combined preparation for treating dilated cardiomyopathy (DCM) in a subject.

As used herein, the term “combination” is intended to refer to all forms of administration that provide a first drug together with a further (second, third.) drug. The drugs may be administered simultaneously, separately or sequentially and in any order. According to the invention, the drug is administered to the subject using any suitable method that enables the drug to reach the chondrocytes of the bone growth plate. In some embodiments, the drug administered to the subject systemically (i.e. via systemic administration). Thus, in some embodiments, the drug is administered to the subject such that it enters the circulatory system and is distributed throughout the body. In some embodiments, the drug is administered to the subject by local administration, for example by local administration to the growing bone.

As used herein, the terms “combined treatment”, “combined therapy” or “therapy combination” refer to a treatment that uses more than one medication. The combined therapy may be dual therapy or bi-therapy.

Accordingly, the invention relates to i) a molecule of CTLA-4 and ii) a classical treatment as a combined preparation according to the invention for simultaneous, separate or sequential use in the method for treating dilated cardiomyopathy (DCM) in a subject.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

In a particular embodiment, the classical treatment refers to radiation therapy, immunotherapy or chemotherapy.

In a particular, the invention relates to i) a molecule of CTLA-4 and ii) a chemotherapy used as a combined preparation for treating dilated cardiomyopathy (DCM) in a subject.

In a particular, the present invention relates to i) a molecule of CTLA-4 and ii) a chemotherapy as a combined preparation according to the invention for simultaneous, separate or sequential use in the method for treating dilated cardiomyopathy (DCM) in a subject.

As used herein, the term “chemotherapy” has its general meaning in the art and refers to the treatment that consists in administering to the patient a chemotherapeutic agent. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000;difluoromethylomithine (DMFO); retinoids such as retinoic acid; capecitabine; and pharmaceutically acceptable salts, acids or derivatives of any of the above.

As used herein, the term “chemotherapeutical drug” refers to any pharmaceutically acceptable composition being therapeutically or pharmaceutically efficient in cancer treatment. A chemotherapeutical drug can be a DNA-alkylating drug, for example.

In a particular, the invention relates to i) a molecule of CTLA-4 and ii) radiotherapy used as a combined preparation for treating dilated cardiomyopathy (DCM) in a subject.

In a particular, the present invention relates to i) a molecule of CTLA-4 and ii) radiotherapy as a combined preparation according to the invention for simultaneous, separate or sequential use in the method for treating dilated cardiomyopathy (DCM) in a subject.

As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In a particular, the invention relates to i) a molecule of CTLA-4 and ii) an immune checkpoint inhibitor used as a combined preparation for treating dilated cardiomyopathy (DCM) in a subject.

In a particular, the present invention relates to i) a molecule of CTLA-4 and ii) an immune checkpoint inhibitor as a combined preparation according to the invention for simultaneous, separate or sequential use in the method for treating dilated cardiomyopathy (DCM) in a subject.

As used herein, the term “immunotherapeutic agent”, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively, the immunotherapeutic treatment may consist of administering the subject with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation).

Interleukins contemplated by the present invention include IL-2, IL-4, IL-11 and IL-12. Examples of commercially available recombinant interleukins include Proleukin® (IL-2;Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention.

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

In some embodiments, the subject will be treated with a CTLA-4 molecule in combination with an immune checkpoint inhibitor.

In some embodiments, the therapy consists of administering to the subject an immune checkpoint inhibitor in combination with CTLA-4 molecule.

In a particular embodiment, i) an immune checkpoint inhibitor and ii) CTLA-4 molecule as a combined preparation according to the invention for simultaneous, separate or sequential use in the method for treating dilated cardiomyopathy in a subject.

As used herein, the term “immune checkpoint inhibitor” (ICI) refers to molecules that totally or partially reduce, inhibit, interfere with or modulate one or more immune checkpoint proteins.

As used herein, the term “immune checkpoint protein” has its general meaning in the art and refers to a molecule that is expressed by T cells in that either turn up a signal (activatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al. 2011. Nature 480:480-489). Examples of activatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-activatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272,has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzyme. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1,Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. No. 7,709,214, U.S. Pat. No. 7,432,059 and U.S. Pat. No. 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolismliu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to -N-(3-bromo-4-fluorophényl)-N′-hydroxy-4-{[2-(sulfamoylamino)-éthyl]amino}-1,2,5-oxadiazole-3 carboximidamide:

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

In a particular, the invention relates to i) molecule of CTLA-4 and ii) an anti-fibrotic agent used as a combined preparation for treating dilated cardiomyopathy (DCM) in a subject.

In a particular, the present invention relates to i) molecule of CTLA-4 and ii) an anti-fibrotic agent as a combined preparation according to the invention for simultaneous, separate or sequential use in the method for treating dilated cardiomyopathy (DCM) in a subject.

As used herein, the term “anti-fibrotic” relates to compound use for the treatment of fibrosis. Such pharmacologically active compound may be compounds for example pirfenidone or nintedanib. Such pharmacologically active compounds may also be substances with a secretolytic, broncholytic and/or anti-inflammatory activity, such as anticholinergic agents, beta-2 mimetics, corticosteroids, PDE-IV inhibitors, p38 MAP kinase inhibitors, MK2inhibitors, galectin inhibitors, NKi antagonists, LTD4 antagonists, EGFR inhibitors, VEGF inhibitors, PDGF inhibitors, FGF inhibitors, TGFbeta inhibitors, LPA1 antagonists, LOXL2 inhibitors, CTGF inhibitors, pentoxyfylline, N-acetylcysteine, anti-IL13 agents, anti IL4 agents, Alpha V integrin inhibitors (including inhibitors of αVβ1, αVβ2, αVβ3, αVβ4, αVβ5, αVβ6, αVβ7, αVβ8, and any combinations thereof), IGF inhibitors, PI3K inhibitors, mTOR inhibitors, JNK inhibitors, pentraxin2 and/or endothelin-antagonists. Other pharmacologically active compounds to be used in combination with the Src kinase inhibitor include compounds with an antifibrotic activity, such as PDE-III inhibitors, combined anti-IL4/13 agents, combined PI3k/mTOR inhibitors, autotaxin inhibitors, P2 X2 antagonists, CTGF antagonists, 5-LO antagonists, leukotriene antagonists, ROCK inhibitors, PDGFR inhibitors (α and/or β) , FGR inhibitors, and/or VEGFR inhibitors. In one embodiment, the anti-fibrotic drug is selected from pirfenidone, or a pharmaceutically acceptable salt thereof, or nintedanib, or a pharmaceutically acceptable salt thereof.

In particular, the invention relates to i) molecule of CTLA-4 and ii) an anti-TNF receptor family used as a combined preparation for treating dilated cardiomyopathy (DCM) in a subject.

In particular, the present invention relates to i) molecule of CTLA-4 and ii) an anti-TNF receptor family as a combined preparation according to the invention for simultaneous, separate or sequential use in the method for treating dilated cardiomyopathy (DCM) in a subject.

As used herein, the terms “tumor necrosis factor receptors” (TNFRs), “Tnf ligand family member” or “TNF family ligand” refers to a proinflammatory cytokine. Cytokines in general, and in particular the members of the TNF ligand family, play a crucial role in the stimulation and coordination of the immune system. At present, nineteen cyctokines have been identified as members of the TNF (tumour necrosis factor) ligand superfamily on the basis of sequence, functional, and structural similarities. All these ligands are type II transmembrane proteins with a C-terminal extracellular domain (ectodomain), N-terminal intracellular domain and a single transmembrane domain. The C-terminal extracellular domain, known as TNF homology domain (THD), has 20-30% amino acid identity between the superfamily members and is responsible for binding to the receptor. The TNF ectodomain is also responsible for the TNF ligands to form trimeric complexes that are recognized by their specific receptors. Members of the TNF ligand family are selected from the group consisting of Lymphotoxin a (also known as LTA or TNFSF1), TNF (also known as TNFSF2), LTβ (also known as TNFSF3), OX40L (also known as TNFSF4), CD40L (also known as CD154 or TNFSF5), FasL (also known as CD95L, CD178 or TNFSF6), CD27L (also known as CD70 or TNFSF7), CD30L (also known as CD153 or TNFSF8), 4-1BBL (also known as TNFSF9), TRAIL (also known as APO2L, CD253 or TNFSF10), RANKL (also known as CD254 or TNFSF11), TWEAK (also known as TNFSF12), APRIL (also known as CD256 or TNFSF13), BAFF (also known as CD257 or TNFSF13B), LIGHT (also known as CD258 or TNFSF14), TL1A (also known as VEGI or TNFSF15), GITRL (also known as TNFSF18), EDA-A1 (also known as ectodysplasin A1) and EDA-A2 (also known as ectodysplasin A2). The term refers to any native TNF family ligand from any vertebrate source, including mammals such as primates (e.g. humans), non-human primates (e.g. cynomolgus monkeys) and rodents (e.g. mice and rats), unless otherwise indicated.

Pharmacologically active anti-TNF compounds comprise but are not limited to Infliximab (Remicade®), Adalimumab (Humira®), Etanercept (Enbrel®), Golimumab (Simponi®), Thalidomide, Certolizumab pegol.

As used the term “A proliferation-inducing ligand” (APRIL), also known as tumor necrosis factor ligand superfamily member 13 (TNFSF13), is a protein of the TNF superfamily recognized by the cell surface receptor TACI. The APRIL has the following Uniprot number: 075888.

In some embodiment, the molecule of CTLA-4 of the present invention is combined with an anti-a proliferation-inducing ligand (APRIL).

In particular, the present invention relates to i) molecule of CTLA-4 and ii) an anti-APRIL used as a combined preparation for treating dilated cardiomyopathy (DCM) in a subject.

In particular, the present invention relates to i) molecule of CTLA-4 and ii) an anti-APRIL as a combined preparation according to the invention for simultaneous, separate or sequential use in the method for treating dilated cardiomyopathy (DCM) in a subject.

In particular, the present invention relates to i) abatacept and ii) an anti-APRIL used as a combined preparation for treating dilated cardiomyopathy (DCM) in a subject.

In particular, the present invention relates to i) abatacept and ii) an anti-APRIL as a combined preparation according to the invention for simultaneous, separate or sequential use in the method for treating dilated cardiomyopathy (DCM) in a subject.

In particular, the present invention relates to i) belatacept and ii) an anti-APRIL used as a combined preparation for treating dilated cardiomyopathy (DCM) in a subject.

In particular, the present invention relates to i) belatacept and ii) an anti-APRIL as a combined preparation according to the invention for simultaneous, separate or sequential use in the method for treating dilated cardiomyopathy (DCM) in a subject.

Members of the anti-a proliferation-inducing ligand (APRIL) are well known in this art and comprise but are not limited to zigakibart (also known as BIO-1301), sibeprenlimab (also known as VIS649) or VIS624.

As used the term “Osteoprotegerin” (OPG), also known as osteoclastogenesis inhibitory factor (OCIF) or tumour necrosis factor receptor superfamily member 11B (TNFRSF11B), is a cytokine receptor of the tumour necrosis factor (TNF) receptor superfamily encoded by the TNFRSF11B gene. OPG binds to TNF-related apoptosis-inducing ligand (TRAIL) and inhibits TRAIL induced apoptosis of specific cells, including tumour cells. The Osteoprotegerin has the following Uniprot number: O00300.

In some embodiment, the molecule of CTLA-4 of the present invention is combined with an anti-Osteoprotegerin (OPG).

In particular, the present invention relates to i) molecule of CTLA-4 and ii) an anti-OPG used as a combined preparation for treating dilated cardiomyopathy (DCM) in a subject.

In particular, the present invention relates to i) molecule of CTLA-4 and ii) an anti-OPG as a combined preparation according to the invention for simultaneous, separate or sequential use in the method for treating dilated cardiomyopathy (DCM) in a subject.

In particular, the present invention relates to i) abatacept and ii) an anti-OPG used as a combined preparation for treating dilated cardiomyopathy (DCM) in a subject.

In particular, the present invention relates to i) abatacept and ii) an anti-OPG as a combined preparation according to the invention for simultaneous, separate or sequential use in the method for treating dilated cardiomyopathy (DCM) in a subject.

In particular, the present invention relates to i) belatacept and ii) an anti-OPG used as a combined preparation for treating dilated cardiomyopathy (DCM) in a subject.

In particular, the present invention relates to i) belatacept and ii) an anti-OPG as a combined preparation according to the invention for simultaneous, separate or sequential use in the method for treating dilated cardiomyopathy (DCM) in a subject.

Members of the anti-Osteoprotegerin (OPG) are well known in this art and comprise but are not limited to anti-OPG antagonist such as anti-OPG antibody.

### Pharmaceutical Composition

The molecule of CTLA-4 for use according to the invention alone and/or combined with classical treatment as described above may be combined with pharmaceutically acceptable excipients, and optionally sustained-release matrices, such as biodegradable polymers, to form pharmaceutical compositions.

Accordingly, in a further aspect, the invention relates to a pharmaceutical composition comprising a molecule of CTLA-4 for dilated cardiomyopathy.

The term “pharmaceutically” or “pharmaceutically acceptable” refers to molecular entities and compositions that do not produce an adverse, allergic or other untoward reaction when administered to a mammal, especially a human, as appropriate.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/ml sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted considering the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

### Method for Screening

In a further aspect, the invention relates to a method of screening a drug suitable for the dilated cardiomyopathy comprising i) providing a test compound and ii) determining the ability of said test compound to activate the expression or activity of CTLA-4.

Any biological assay well known in the art could be suitable for determining the ability of the test compound to activate the activity or expression of CTLA-4. In some embodiments, the assay first comprises determining the ability of the test compound to bind to CTLA-4. In some embodiments, a population of cardiac cells then contacted and activated so as to determine the ability of the test compound to activate the activity or expression of CTLA-4. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of activating the activity or expression of CTLA-4, as determined using in vitro methods described herein, are likely to exhibit similar modulatory capacity in applications in vivo. Typically, the test compound is selected from the group consisting of peptides, petptidomimetics, small organic molecules, antibodies (e.g. intraantibodies), aptamers or nucleic acids. For example, the test compound according to the invention may be selected from a library of compounds previously synthesised, or a library of compounds for which the structure is determined in a database, or from a library of compounds that have been synthesised de novo.

The invention will be further illustrated by the following figures and examples. However, these examples and figures should not be interpreted in any way as limiting the scope of the present invention.

### FIGURES

FIG. 1: Comparison with TAC C57 at 6 weeks post-TAC. (A) End-systolic parameters (B) End-diastolic parameters. One injection AAV1/2-CTLA-4-Fc intramuscular 24 h after TAC (C) Cardiac total IgG (8 weeks post TAC). PWT: posterior wall thickness—AWT: anterior wall thickness—ES: end-sytolic-ED: end—diastolic—LVESD: Left ventricular end-systolic diameter—LVEDD: Left ventricular end-diastolic diameter.

### EXAMPLE:

### Results

Data obtained by qPCR reveal a significant decrease in the expression of CTLA-4 in the Balb/c strain compared to the C57 strain. It should be noted that CTLA-4 (co-inhibition) binds preferentially to CD80/86, compared to CD28 (co-stimulation). However, although a similar increase in the expression of CD80/86 is observed in the two genetic backgrounds, only CTLA-4 is more strongly expressed in C57, which results in a stronger inhibition of the pathway of CD28 signaling after TAC versus Balb/C. These experimental results are very interesting in view of the clinical literature since a CTLA-4 polymorphism is associated with a form of dilated cardiomyopathy (DCM) in humans [1].

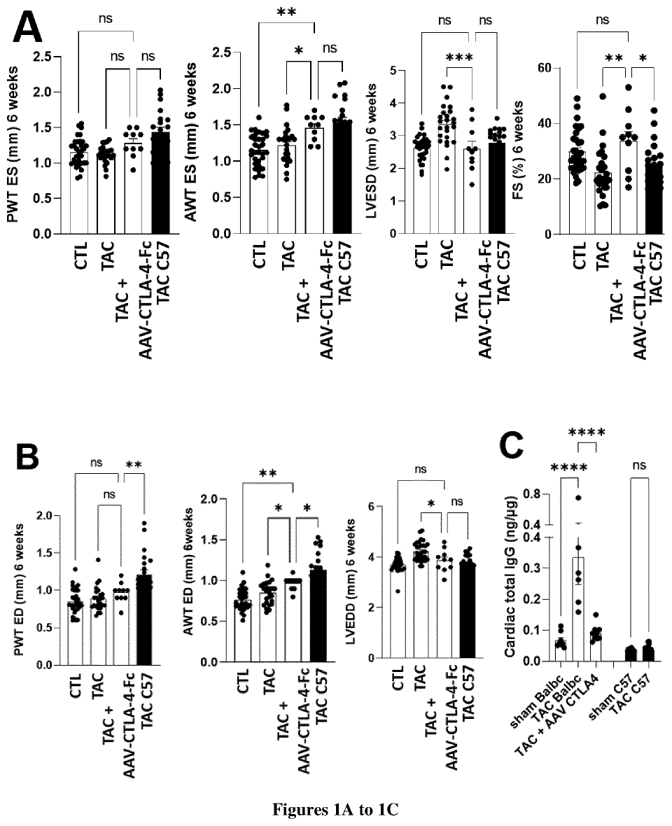

In this context, the CTLA-4 pathway is pursued to highlight its involvement in the progression to dilated cardiomyopathy via the B lymphocyte response. Indeed, Balb/c cells show a deficit B response associated with a CTLA-4 defect, compared to the C57. However, in the TAC model carried out in C57, it has been shown that blocking co-signaling by CD80-CD86 by immunotherapy via abatacept reduces the progression and severity of cardiac dysfunction via the production of IL-10 by B lymphocytes essentially associated with preservation of cardiomyocytes [2]. In particular, our analyzes of data published in scRNAseq in this model [3], of immune cells infiltrating the heart reveals that in C57, the genes of the CTLA-4 pathway are highly enriched and that CTLA-4 is expressed mainly by Tregs and activated B cells [3]. Additionally, mice carrying a CTLA-4 deletion in B cells have been shown to selectively develop autoantibodies, follicular helper T cells (Tfh) and germinal centers in the spleen, as well as autoimmune pathologies with age[4]. This alteration in immune homeostasis results from dysfunctional B-1a cells upon loss of CTLA-4. This suggests that in the C57 strain, the higher expression of CTLA-4 will protect the heart against antibody-mediated immunity, despite the increase in cardiac B cell counts (regulatory B cells). On the other hand, the absence of CTLA-4 signaling in Balb/c could favors antibodies-secreting B cells (FIG. 1C). On the other hand, after TAC, the cardiac levels of another co-signalling molecule, α (lymphotoxin-α), is increased in particular in Balb/c, where it could promote lymphangiogenesis[5] allowing the evacuation of lymphocytes B.

Post-TAC Balb/c show left ventricle (LV) dilation at six weeks associated with a significantly decreased fractional shortening reflecting the eccentric hypertrophic phenotype. CTLA-4-Fc induces concentric hypertrophy of the ventricular walls (from 3 weeks) which is intermediate to that observed in TAC C57 at 6 weeks but which is not associated with a reduction in the fraction of narrowing of the LV contrary to C57, which therefore suggests a beneficial hypertrophy and preserved cardiac function (FIGS. 1A and 1B). Furthermore, CTLA-4-Fc treatment blunts cardiac humoral response (FIG. 1C)

All this element suggests that the use of CLTA-4 can treat dilated cardiomyopathy.

### REFERENCES

Throughout this application, various references describe the state of the art to which this invention pertains. The disclosures of these references are hereby incorporated by reference into the present disclosure.
cardiomyopathy », Eur. J. Hum. Genet., vol. 18, no 6, p. 694-699, juin 2010, doi: 10.1038/ejhg.2010.3.
[2] M. Kallikourdis et al., «T cell costimulation blockade blunts pressure overload-induced heart failure », Nat. Commun., vol. 8, p. 14680, mars 2017, doi: 10.1038/ncomms14680.
[3] E. Martini et al., «Single-Cell Sequencing of Mouse Heart Immune Infiltrate in Pressure Overload-Driven Heart Failure Reveals Extent of Immune Activation », Circulation, vol. 140, no 25, p. 2089-2107, déc. 2019, doi: 10.1161/CIRCULATIONAHA.119.041694.
[4] Y. Yang et al., «CTLA-4 expression by B-1a B cells is essential for immune tolerance », Nat. Commun., vol. 12, no 1, p. 525, janv. 2021, doi: 10.1038/s41467-020-20874-x.
[5] R. H. Mounzer et al., «Lymphotoxin-alpha contributes to lymphangiogenesis », Blood, vol. 116, no 12, p. 2173-2182, sept. 2010, doi: 10.1182/blood-2009-12-256065.

1. A method of treating dilated cardiomyopathy (DCM) in a subject in need thereof comprising a step of administering to said subject a therapeutically effective amount of a cytotoxic T-lymphocyte-associated protein 4 (CTLA-4) molecule.

2. The method of claim 1 wherein the molecule of CTLA-4 is a small organic molecule.

3. The method of claim 1 wherein the molecule of CTLA-4 is abatacept.

4. The method of claim 1 wherein the molecule of CTLA-4 is belatacept.

5. The method of claim 1 wherein the molecule of CTLA-4 is combined with radiation therapy.

6. The method of claim 1 wherein the molecule of CTLA-4 is combined with immunotherapy.

7. The method of claim 1 wherein the molecule of CTLA-4 is combined with chemotherapy.

8. The method of claim 1 wherein the molecule of CTLA-4 is combined with an anti-fibrotic agent.

9. The method of claim 1 wherein the molecule of CTLA-4 is combined with an anti-TNF receptor family.

10. The method of claim 1 wherein the molecule of CTLA-4 is combined with anti-a proliferation-inducing ligand (APRIL).

11. The method of claim 1 wherein the molecule of CTLA-4 is combined with anti-Osteoprotegerin (OPG).

12. A method for modulating cardiac remodeling in a subject in need thereof comprising a step of administering to said subject a therapeutically effective amount of a cytotoxic T-lymphocyte-associated protein 4 (CTLA-4) molecule.

13. A pharmaceutical composition comprising a molecule of CTLA-4.

14. A method of screening a drug suitable for treating dilated cardiomyopathy (DCM) comprising i) providing a test compound and ii) determining the ability of said test compound to activate the expression or activity of CTLA-4.
